(19) United States
(12) Patent Application Publication
(10) Pub. No.: US 2026/0128144 A1
(43) Pub. Date: May 7, 2026

(54) Systems and Methods for Pharmacotherapeutic Intervention in Obesity Treatment Targeting Multiple Neurophysiological Mechanisms Using Patient Phenotyping Modeling

(71) Applicant: Intellihealth, Inc., New Cannan, CT (US)

(72) Inventors: David H. Bass, San Francisco, CA (US); Leon Igel, Brookville, NY (US); Christina Lorenzo, Steamboat Springs, CO (US); Cheryl Pegus, Henderson, NV (US); Nathan Lesch, Minneapolis, MN (US); Guadalupe Minero, Poway, CA (US); Venkateswaran Suriyanarayanan, San Jose, CA (US)

(21) Appl. No.: 19/375,074

(22) Filed: Oct. 30, 2025

Related U.S. Application Data
(60) Provisional application No. 63/716,064, filed on Nov. 4, 2024.

(51) Int. Cl. G16H 20/10; G16H 10/60; G16H 15/00; G16H 20/30; G16H 50/30

(52) U.S. Cl. CPC G16H 20/10; G16H 10/60; G16H 15/00; G16H 20/30; G16H 50/30

(57) ABSTRACT

Systems and methods for pharmacotherapeutic intervention in obesity treatment are disclosed, targeting multiple neurophysiological mechanisms of appetite and weight regulation. The described technology utilizes patient phenotyping, including body mass index (BMI), medical history, and laboratory data, to classify patients and select individualized therapies. Solutions include administering non-GLP-1 agonist anti-obesity medications, dual therapies, or agents targeting GLP-1 and GIP receptors, combined with lifestyle modifications (diet, physical activity). The approach aims to achieve significant and durable weight loss while minimizing compensatory physiological responses, with periodic re-evaluation and treatment updates. An AI-driven decision support tool may be used for personalized recommendations. Principal uses include treating patients with varying BMI ranges and comorbidities, including type 2 diabetes mellitus, to improve metabolic health and quality of life. The described method is characterized by selecting and administering pharmacotherapy based on patient classification and ongoing assessment.

### CROSS REFERENCE TO RELATED APPLICATIONS

This application claims the priority benefit of U.S. Provisional Patent Application Ser. No. 63/716,064, filed on Nov. 4, 2024, and titled “Systems and Methods for Pharmacotherapeutic Intervention in Obesity Treatment Targeting Multiple Neurophysiological Mechanisms Using Patient Phenotyping Modeling.” The aforementioned disclosure is hereby incorporated by reference in its entirety for all purposes.

### FIELD OF THE TECHNOLOGY

This disclosure relates generally to medical technologies, specifically pharmacotherapeutic interventions for obesity treatment and obesity-related comorbidities using patient phenotyping modeling

### BACKGROUND

In recent years, obesity and related metabolic disorders have become a significant concern globally. Medical technologies aimed at body-weight regulation span a wide range of interventions, including dietary, behavioral, surgical, and pharmacological strategies. Among these, pharmacotherapeutic approaches seek to modulate neurophysiological systems that govern appetite, satiety, energy expenditure, and nutrient absorption. Advances in diagnostic methods and patient-phenotyping tools have raised the possibility of tailoring treatments to individual physiological and genetic profiles. Nonetheless, the intricate and dynamic nature of weight-regulation mechanisms poses ongoing challenges for clinicians and researchers striving to translate scientific insights into safe and effective therapies.

Efforts to treat obesity focus not only on achieving initial weight reduction but also on sustaining long-term improvements in metabolic health and quality of life. Desired outcomes include lowering cardiovascular risk factors, reducing incidence of type 2 diabetes and fatty liver disease, and alleviating obesity-associated respiratory and musculoskeletal complications. In clinical practice, interventions are expected to harmonize pharmacological regimens with lifestyle modifications, such as tailored dietary plans, structured physical activity, and behavior support, to optimize adherence and minimize adverse effects. Health care providers aim to deliver individualized guidance based on patient history, comorbidities, and treatment responsiveness, with the primary objective of improving overall health metrics while maintaining patient safety and tolerance.

Traditional approaches for weight management, including caloric restriction and exercise, frequently fail to yield sustained results. Physiological safeguards designed to preserve energy stores activate in response to reduced intake and increased activity, slowing basal metabolic rate and heightening appetite signals. These compensatory adaptations often manifest as increased hunger, reduced satiety, and metabolic adaptation that blunts energy expenditure. As a result, patients commonly experience plateaus or regain lost weight, which can undermine motivation and long-term success. Moreover, variability in individual responses to behavioral interventions further complicates treatment planning, highlighting limitations in the capacity of conventional regimens to produce durable weight loss across diverse patient populations.

Pharmacological solutions that target a single neurophysiological pathway have demonstrated modest efficacy, yet their benefits frequently diminish over time. When one regulatory mechanism is suppressed or enhanced, alternative circuits may be recruited to re-establish homeostasis, thereby attenuating initial weight reductions. This counter-regulatory phenomenon underscores the complexity of appetite and energy-balance systems, which involve overlapping networks of hormones, neurotransmitters, and signaling cascades. Additionally, individual variations in receptor profiles, hormonal set points, and metabolic rate contribute to inconsistent clinical outcomes. As a result, there remains a pressing need for therapeutic strategies capable of simultaneously addressing multiple weight-regulating pathways, thereby minimizing compensatory responses, and improving the durability of treatment effects.

### SUMMARY

Some embodiments relate to a method of selecting a pharmacotherapeutic intervention for treatment of obesity targeting multiple dedicated neurophysiological mechanisms of appetite and weight regulation to achieve significant and durable weight loss for a classified patient with a Body Mass Index (BMI) of 27 to 29.9, the method including: receiving patient data including at least body-mass-index (BMI), medical history, laboratory data, and prior medication response; classifying a patient into a predefined BMI range using the patient data, the predefined BMI range being a Body Mass Index (BMI) of 27 to 29.9 thereby the classified patient being classified as overweight; determining, for the classified patient, an absence of type-2 diabetes mellitus using the patient data; receiving a lifestyle modification confirmation for the classified patient, the lifestyle modification confirmation for the classified patient being that the classified patient makes a lifestyle modification; administering a therapeutically effective amount of a non-glucagon-like peptide-1 (non-GLP-1) agonist anti-obesity medication to the classified patient, the administering the therapeutically effective amount of the non-GLP-1 agonist anti-obesity medication to the classified patient being a particular prophylaxis targeting multiple dedicated neurophysiological mechanisms that is an obesity treatment for patients having a Body Mass Index (BMI) of approximately 27 to 29.9 in combination with the lifestyle modification confirmation; and re-evaluating the classified patient and updating a medical treatment plan of the classified patient based on the re-evaluating.

In some embodiments the non-glucagon-like peptide-1 (non-GLP-1) agonist anti-obesity medication is at least one of: metformin, topiramate, zonisamide, bupropion, naltrexone, phentermine, and orlistat.

In some embodiments the lifestyle modification confirmation includes confirmation that the classified patient adopts a hypocaloric diet or increases physical activity to a target of at least 150 minutes per week.

In some embodiments re-evaluating the classified patient includes measuring a body weight of the classified patient and reassessing body-mass-index (BMI) after a three-month interval.

In some embodiments determining the absence of type-2 diabetes mellitus includes analyzing a glycated hemoglobin (HbA1c) level of the classified patient to confirm that the glycated hemoglobin (HbA1c) level is below 6.5%.

Some embodiments relate to a method of selecting a pharmacotherapeutic intervention for treatment of obesity targeting multiple dedicated neurophysiological mechanisms of appetite and weight regulation to achieve significant and durable weight loss for a classified patient with a Body Mass Index (BMI) of 30 to 34.9, the method including: receiving patient data including at least body-mass-index (BMI), medical history, laboratory data, and prior medication response; classifying a patient into a predefined BMI range using the patient data, the predefined BMI range being a Body Mass Index (BMI) of 30 to 34.9 thereby the classified patient being classified in class 1 of obesity; determining, for the classified patient, an absence of type-2 diabetes mellitus using the patient data; determining, for the classified patient, an absence of history or current diagnosis of myocardial infarction, stroke, chronic heart failure, hypertension, obstructive sleep apnea, or non-alcoholic fatty liver disease; receiving a lifestyle modification confirmation for the classified patient, the lifestyle modification confirmation for the classified patient being that the classified patient makes a lifestyle modification; administering a therapeutically effective amount of dual therapy non-glucagon-like peptide-1 (non-GLP-1) agonist anti-obesity medications to the classified patient, the administering the therapeutically effective amount of the dual therapy non-GLP-1 agonist anti-obesity medications to the classified patient being a particular prophylaxis targeting multiple dedicated neurophysiological mechanisms that is an obesity treatment for patients having a Body Mass Index (BMI) of approximately 30 to 34.9 in combination with the lifestyle modification confirmation; and re-evaluating the patient and updating a medical treatment of the patient based on the re-evaluating.

In some embodiments the dual therapy non-glucagon-like peptide-1 (non-GLP-1) agonist anti-obesity medications include a combination selected from the group consisting of metformin and bupropion, metformin and topiramate, bupropion and topiramate, bupropion and naltrexone, phentermine and topiramate, and phentermine and metformin.

In some embodiments the lifestyle modification includes adoption of an increase in physical activity for the classified patient to a target of at least 150 minutes per week.

In some embodiments the re-evaluating the classified patient includes measuring a body weight of the classified patient and reassessing body-mass-index (BMI) after a three-month interval.

Some embodiments relate to a method of selecting a pharmacotherapeutic intervention for treatment of obesity targeting multiple dedicated neurophysiological mechanisms of appetite and weight regulation to achieve significant and durable weight loss for a classified patient with a Body Mass Index (BMI) of 35 to 39.9, the method including: receiving patient data including at least body-mass-index (BMI), medical history, laboratory data, and prior medication response; classifying a patient into a predefined BMI range using the patient data, the predefined BMI range being a Body Mass Index (BMI) of 35 to 39.9 thereby the classified patient being classified in class 2 of obesity; determining, for the classified patient, an absence of type-2 diabetes mellitus using the patient data; determining, for the classified patient, an absence of history or current diagnosis of myocardial infarction, stroke, chronic heart failure, hypertension, obstructive sleep apnea, or non-alcoholic fatty liver disease; receiving a lifestyle modification confirmation for the classified patient, the lifestyle modification confirmation for the classified patient being that the classified patient makes a lifestyle modification; administering a therapeutically effective amount of dual therapy non-glucagon-like peptide-1 (non-GLP-1) agonist anti-obesity medications to the classified patient, the administering the therapeutically effective amount of the dual therapy non-GLP-1 agonist anti-obesity medications to the classified patient being a particular prophylaxis targeting multiple dedicated neurophysiological mechanisms that is an obesity treatment for patients having a Body Mass Index (BMI) of approximately 30 to 34.9 in combination with the lifestyle modification confirmation; and re-evaluating the patient and updating a medical treatment of the patient based on the re-evaluating.

In some embodiments the dual therapy non-glucagon-like peptide-1 (non-GLP-1) agonist anti-obesity medications include a combination selected from the group consisting of metformin and bupropion, metformin and topiramate, bupropion and topiramate, bupropion and naltrexone, phentermine and topiramate, and phentermine and metformin.

In some embodiments the lifestyle modification includes adoption of an increase in physical activity for the classified patient to a target of at least 150 minutes per week.

In some embodiments the re-evaluating the classified patient includes measuring a body weight of the classified patient and reassessing body-mass-index (BMI) after a three-month interval.

Some embodiments relate to a method of selecting a pharmacotherapeutic intervention for treatment of obesity targeting multiple dedicated neurophysiological mechanisms of appetite and weight regulation to achieve significant and durable weight loss for a classified patient with a Body Mass Index (BMI) of 40 or higher, the method including: receiving patient data including at least body-mass-index (BMI), medical history, laboratory data, and prior medication response; classifying a patient into a predefined BMI range using the patient data, the predefined BMI range being a Body Mass Index (BMI) of 40 or higher thereby the classified patient being classified in class 3 of obesity; determining, for the classified patient, an absence of type-2 diabetes mellitus using the patient data; receiving a lifestyle modification confirmation for the classified patient, the lifestyle modification confirmation for the classified patient being that the classified patient makes a lifestyle modification; determining, for the classified patient, a history or current diagnosis of myocardial infarction, stroke, chronic heart failure, hypertension, obstructive sleep apnea, or non-alcoholic fatty liver disease; administering a therapeutically effective amount of dual therapy non-glucagon-like peptide-1 (non-GLP-1) agonist anti-obesity medications or Gastric Inhibitory Polypeptide (GIP) agonist to the classified patient, the administering the therapeutically effective amount of the dual therapy non-GLP-1 agonist anti-obesity medications or Gastric Inhibitory Polypeptide (GIP) agonist being a particular prophylaxis targeting multiple dedicated neurophysiological mechanisms that is an obesity treatment for patients having a Body Mass Index (BMI) of 40 or higher in combination with the lifestyle modification confirmation; and re-evaluating the patient and updating a medical treatment of the patient based on the re-evaluating.

In some embodiments the dual therapy non-glucagon-like peptide-1 (non-GLP-1) agonist anti-obesity medications include a combination selected from the group consisting of metformin and bupropion, metformin and topiramate, bupropion and topiramate, bupropion and naltrexone, phentermine and topiramate, and phentermine and metformin.

In some embodiments the lifestyle modification includes adoption of an increase in physical activity for the classified patient to a target of at least 150 minutes per week.

In some embodiments the re-evaluating the classified patient includes measuring a body weight of the classified patient and reassessing body-mass-index (BMI) after a three-month interval.

In some aspects, the techniques described herein relate to a method of pharmacotherapeutic intervention for treatment of obesity targeting multiple dedicated neurophysiological mechanisms of appetite and weight regulation to achieve significant and durable weight loss for a patient that has a diagnosis of type 2 diabetes mellitus, the method including: ascertaining a patient has a diagnosis of type 2 diabetes mellitus; determining a recent hemoglobin A1c level to be either below 9% or greater than 9% for the patient; determining a recent hemoglobin A1c level is greater than 9%; determining if a most recent c-peptide level is either low or normal/high; determining the most recent c-peptide level is normal/high; determining if the patient is on metformin or contraindicated for metformin; and determining the recent hemoglobin A1c level is between 9% to 10%; and administering a therapeutically effective amount of medications to the patient targeting either Glucagon-Like Peptide-1 (GLP-1) receptors or both Glucagon-Like Peptide-1 (GLP-1) and Gastric Inhibitory Polypeptide (GIP) receptors, the administering being a particular prophylaxis targeting multiple dedicated neurophysiological mechanisms that is a specific obesity treatment for patients having a diagnosis of type 2 diabetes mellitus and a recent hemoglobin A1c level between 9% to 10%.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure, and explain various principles and advantages of those embodiments.

FIG. 1 illustrates a method of pharmacotherapeutic intervention for treatment of obesity targeting multiple dedicated neurophysiological mechanisms of appetite and weight regulation to achieve significant and durable weight loss for a patient with a body mass index of approximately 27 to 29.9, according to embodiments of the present technology.

FIG. 2 illustrates a method of pharmacotherapeutic intervention for treatment of obesity targeting multiple dedicated neurophysiological mechanisms of appetite and weight regulation to achieve significant and durable weight loss for a patient with a body mass index of approximately 30 to 34.9, according to embodiments of the present technology.

FIG. 3 illustrates a method of pharmacotherapeutic intervention for treatment of obesity targeting multiple dedicated neurophysiological mechanisms of appetite and weight regulation to achieve significant and durable weight loss for a patient with a body mass index of approximately 35 to 39.9, according to embodiments of the present technology.

FIG. 4 illustrates a method of pharmacotherapeutic intervention for treatment of obesity targeting multiple dedicated neurophysiological mechanisms of appetite and weight regulation to achieve significant and durable weight loss for a patient with a body mass index of approximately 40 or higher, according to embodiments of the present technology.

FIG. 5 illustrates a method of pharmacotherapeutic intervention for treatment of obesity targeting multiple dedicated neurophysiological mechanisms of appetite and weight regulation to achieve significant and durable weight loss for a patient with a diagnosis of type 2 diabetes mellitus, according to embodiments of the present technology.

FIG. 6 illustrates dual-therapy and mono-therapy options for a method of pharmacotherapeutic intervention for treatment of obesity targeting multiple dedicated neurophysiological mechanisms of appetite and weight regulation to achieve significant and durable weight loss for a patient, according to embodiments of the present technology.

FIG. 7 illustrates dual-therapy and mono-therapy options for a method of pharmacotherapeutic intervention for treatment of obesity targeting multiple dedicated neurophysiological mechanisms of appetite and weight regulation to achieve significant and durable weight loss for a patient, according to embodiments of the present technology.

FIG. 8 illustrates an exemplary computer system that may be used to implement embodiments of the present technology.

### DETAILED DESCRIPTION

In the following description, for purposes of explanation, numerous specific details are set forth in order to provide a thorough understanding of the disclosure. It will be apparent, however, to one skilled in the art, that the disclosure may be practiced without these specific details. In other instances, structures and devices may be shown in block diagram form only in order to avoid obscuring the disclosure. It should be understood, that the disclosed embodiments are merely exemplary of the invention, which may be embodied in multiple forms. Those details disclosed herein are not to be interpreted in any form as limiting, but as the basis for the claims.

The following detailed description is provided to enable those skilled in the art to make and use the embodiments of the present subject matter. The description is intended to be illustrative and is not to be construed as limiting the scope of the subject matter in any way. While specific details are provided to facilitate a comprehensive understanding of the subject matter, those skilled in the art will recognize that the subject matter may be practiced without some of these specific details. For example, well-known methods, procedures, and components are not described in detail to avoid unnecessarily obscuring the subject matter.

The present disclosure pertains to the field of medical technologies, particularly pharmacotherapeutic interventions for addressing obesity and associated comorbidities. The described approach utilizes patient phenotyping and advanced modeling to address multiple neurophysiological mechanisms involved in appetite and weight regulation. It is to be understood that the embodiments described herein are provided as illustrations, and various modifications, substitutions, and rearrangements of components or steps may be implemented without deviating from the principles and scope of the described subject matter. The claims appended hereto define the boundaries of the described subject matter, and the examples provided are not intended to restrict the scope of those claims.

Obesity treatment presents significant challenges due to the complex interplay of multiple overlapping and redundant systems that regulate body weight. Traditional methods such as exercise and hypocaloric diets often prove insufficient, as the body employs mechanisms to preserve fat mass, including slowing basal metabolic rate, increasing appetite, and decreasing satiety. These physiological responses complicate efforts to achieve and maintain weight loss.

Pharmacological approaches targeting a single neurophysiological pathway frequently fail to produce substantial and lasting weight loss. This failure occurs because secondary systems activate to re-stabilize body weight, a phenomenon known as metabolic adaptation. Consequently, there is a recognized need for therapeutic strategies that address multiple weight-regulating systems simultaneously. Such approaches may prevent counter-regulation and enhance the effectiveness of obesity treatments.

The present technology in various embodiments introduces methods of pharmacotherapeutic intervention for obesity treatment that targets multiple dedicated neurophysiological mechanisms of appetite and weight regulation. This approach aims to achieve significant and durable weight loss by considering the individual phenotype of the patient. The method involves a combination prescribing algorithm that synergistically targets various neurophysiological pathways, informed by a Health Risk Assessment questionnaire. This enables the evaluation of currently prescribed medications, medical diagnoses, and available laboratory data to provide individualized pharmacotherapeutic recommendations.

Various embodiments of the present technology include a combination prescribing algorithm that synergistically targets multiple dedicated neurophysiological mechanisms of appetite and weight regulation to achieve significant and durable weight loss. The present technology includes a medication decision support (MDS) tool, informed by a Health Risk Assessment (HRA) questionnaire, which assesses the individual phenotype of the patient, evaluates the currently prescribed medications and medical diagnoses, integrates the available laboratory data, and assesses the absolute and relative contraindications to provide individualized recommendations for the most appropriate pharmacotherapeutic intervention.

Obesity and the associated comorbidities, such as type 2 diabetes mellitus, cardiovascular disease, and non-alcoholic fatty liver disease, present significant challenges due to the complex interplay of neurophysiological mechanisms regulating appetite, energy balance, and weight. Traditional interventions, including caloric restriction, physical activity, and pharmacological treatments targeting single pathways, often fail to achieve sustained weight loss due to compensatory physiological responses, such as increased appetite and reduced metabolic rate. The described system and method address these challenges by providing a personalized pharmacotherapeutic intervention that targets multiple neurophysiological mechanisms simultaneously, leveraging patient phenotyping and advanced modeling to enhance treatment outcomes.

In various embodiments the present technology are employed in clinical settings to treat patients with varying body mass index (BMI) ranges and comorbidities. The system utilizes a combination prescribing algorithm informed by patient-specific data, including BMI, medical history, laboratory results, and prior medication responses. The method involves administering tailored pharmacological therapies, such as non-GLP-1 agonist anti-obesity medications, dual therapies, or agents targeting GLP-1 and GIP receptors, in conjunction with lifestyle modifications like dietary adjustments and increased physical activity. Treatment protocols are further refined through periodic re-evaluation of patient progress, including weight, BMI, and other metabolic health indicators, typically at three-month intervals. The disclosed approach also contemplates the use of an AI-driven decision support tool to enhance the precision of pharmacotherapy recommendations. Alternative configurations, such as varying drug combinations or dosing regimens, are encompassed within the scope of the disclosed methods, ensuring adaptability to diverse patient needs and clinical scenarios. Representative performance results demonstrate significant and durable weight loss, improved metabolic health, and enhanced quality of life, as further illustrated in the accompanying examples and figures.

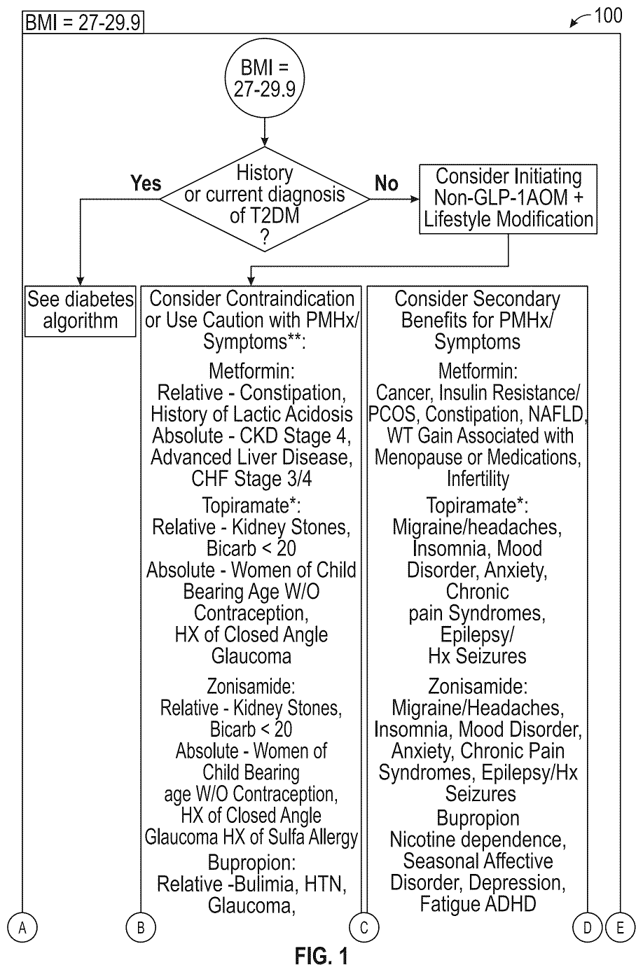

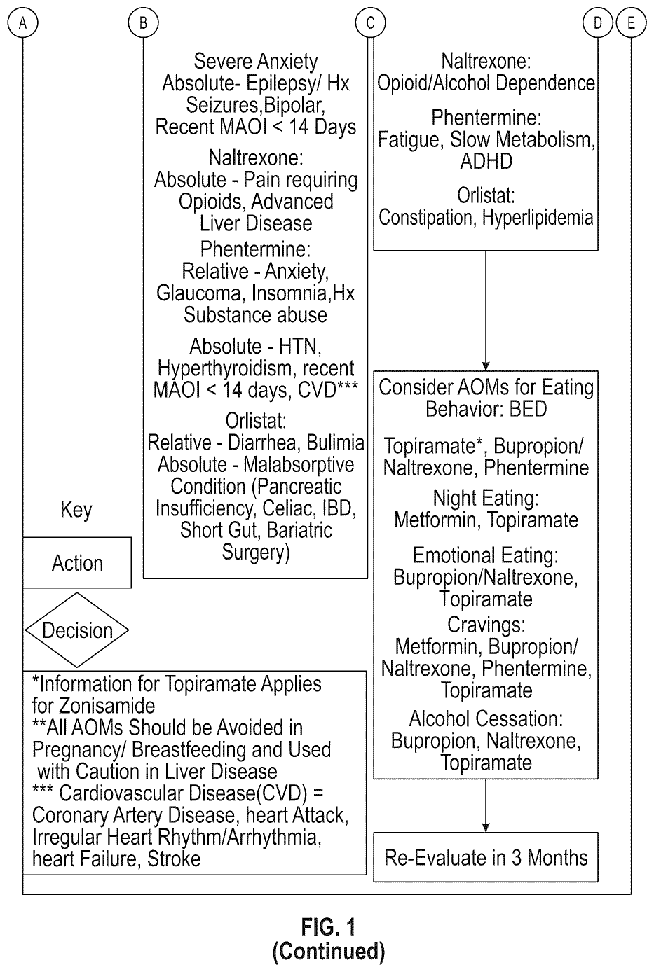

FIG. 1 illustrates a method 100 of pharmacotherapeutic intervention for treatment of obesity targeting multiple dedicated neurophysiological mechanisms of appetite and weight regulation to achieve significant and durable weight loss for a patient with a body mass index of approximately 27 to 29.9, according to embodiments of the present technology. FIG. 1 illustrates the method 100 of pharmacotherapeutic intervention for treatment of obesity targeting multiple dedicated neurophysiological mechanisms of appetite and weight regulation to achieve significant and durable weight loss for a patient, the method comprising: ascertaining a patient has a body mass index of approximately 27 to 29.9; ascertaining the patient does not have a history or current diagnosis of type 2 diabetes mellitus; administering a non-GLP-1 agonist anti-obesity medication to the patient; receiving lifestyle modification information that the patient makes a lifestyle modification, while taking into account a past medical history of the patient with respect to contraindications and secondary benefits of medications; receiving an eating behavior confirmation that the patient changes an eating behavior; and re-evaluating the patient and updating a medical treatment of the patient based on the re-evaluating.

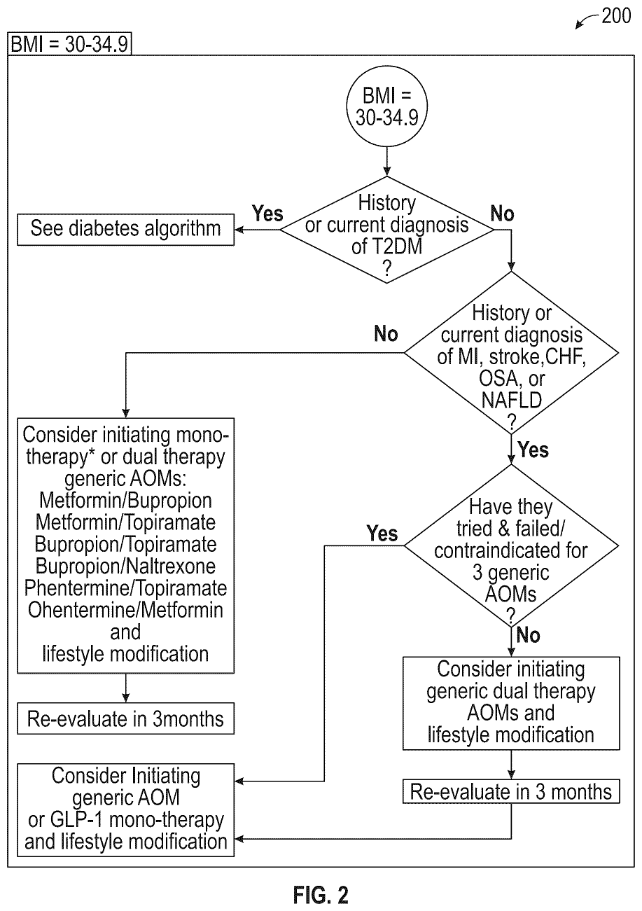

FIG. 2 illustrates a method 200 of pharmacotherapeutic intervention for treatment of obesity targeting multiple dedicated neurophysiological mechanisms of appetite and weight regulation to achieve significant and durable weight loss for a patient with a body mass index of approximately 30 to 34.9, according to embodiments of the present technology. FIG. 2 illustrates the method 200 of pharmacotherapeutic intervention for treatment of obesity targeting multiple dedicated neurophysiological mechanisms of appetite and weight regulation to achieve significant and durable weight loss for a patient, the method comprising: ascertaining a patient has a body mass index of approximately 30 to 34.9; ascertaining the patient does not have a history or current diagnosis of type 2 diabetes mellitus; ascertaining if the patient has a history or current diagnosis of myocardial infarction, stroke, chronic heart failure, hypertension, obstructive sleep apnea, or non-alcoholic fatty liver disease; if the patient has the history or current diagnosis of myocardial infarction, stroke, chronic heart failure, hypertension, obstructive sleep apnea, or non-alcoholic fatty liver disease, determining if the patient tried and failed (or has contraindications to use of) three generic anti-obesity medications; if the patient tried and failed (or has contraindications to use of) three generic anti-obesity medications, either initiate a fourth generic anti-obesity medication or a Glucagon-Like Peptide-1 (GLP-1) in tandem with lifestyle modification; if the patient does not have the history or current diagnosis of myocardial infarction, stroke, chronic heart failure, hypertension, obstructive sleep apnea, or non-alcoholic fatty liver disease, initiate generic dual therapy anti-obesity medications and a lifestyle modification; if the patient does not have the history or current diagnosis of myocardial infarction, stroke, chronic heart failure, hypertension, obstructive sleep apnea, or non-alcoholic fatty liver disease, initiate mono-therapy or dual therapy generic anti-obesity medications and a lifestyle change; and re-evaluating the patient and updating a medical treatment of the patient based on the re-evaluating.

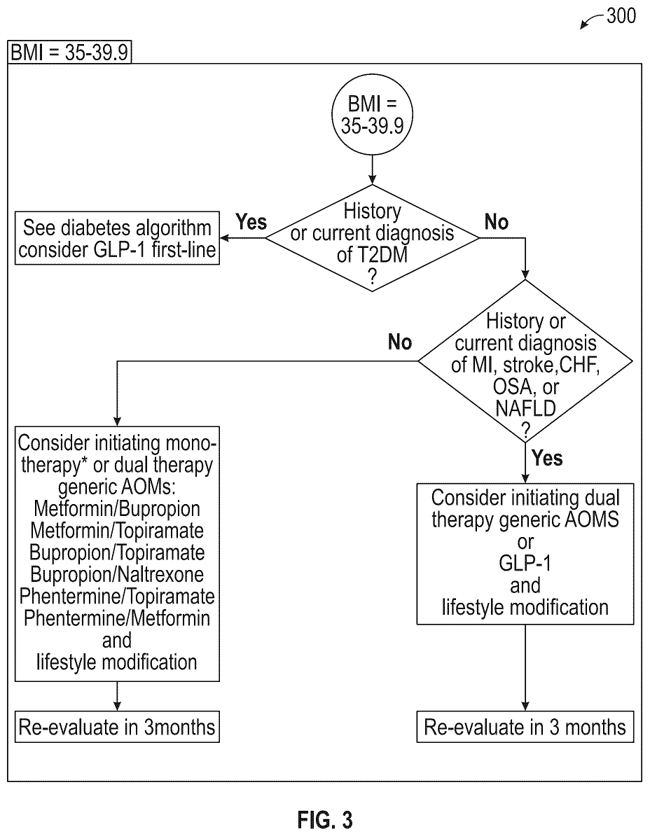

FIG. 3 illustrates a method 300 of pharmacotherapeutic intervention for treatment of obesity targeting multiple dedicated neurophysiological mechanisms of appetite and weight regulation to achieve significant and durable weight loss for a patient with a body mass index of approximately 35 to 39.9, according to embodiments of the present technology. FIG. 3 illustrates the method 300 of pharmacotherapeutic intervention for treatment of obesity targeting multiple dedicated neurophysiological mechanisms of appetite and weight regulation to achieve significant and durable weight loss for a patient, the method comprising: ascertaining a patient has a body mass index of approximately 35 to 39.9; ascertaining the patient does not have a history or current diagnosis of type 2 diabetes mellitus; ascertaining if the patient has a history or current diagnosis of myocardial infarction, stroke, chronic heart failure, hypertension, obstructive sleep apnea, or non-alcoholic fatty liver disease; if the patient has the history or current diagnosis of myocardial infarction, stroke, chronic heart failure, hypertension, obstructive sleep apnea, or non-alcoholic fatty liver disease, initiate dual therapy generic anti-obesity medications or a Glucagon-Like Peptide-1 (GLP-1) and a lifestyle modification; if the patient does not have the history or current diagnosis of myocardial infarction, stroke, chronic heart failure, hypertension, obstructive sleep apnea, or non-alcoholic fatty liver disease, initiate dual therapy generic anti-obesity medications and a lifestyle modification; re-evaluating the patient and updating a medical treatment of the patient based on the re-evaluating.

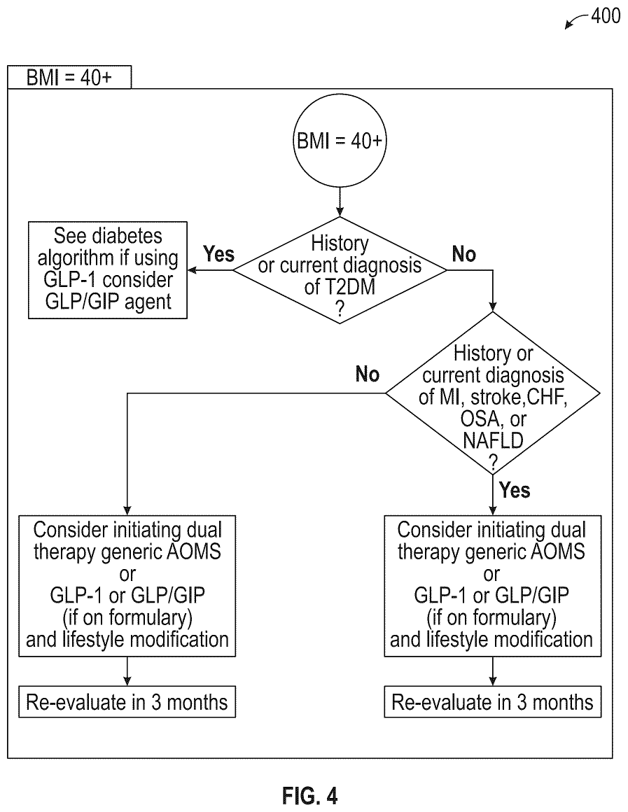

FIG. 4 illustrates a method 400 of pharmacotherapeutic intervention for treatment of obesity targeting multiple dedicated neurophysiological mechanisms of appetite and weight regulation to achieve significant and durable weight loss for a patient with a body mass index of approximately 40 or higher, according to embodiments of the present technology. FIG. 4 illustrates the method 400 of pharmacotherapeutic intervention for treatment of obesity targeting multiple dedicated neurophysiological mechanisms of appetite and weight regulation to achieve significant and durable weight loss for a patient, the method comprising: ascertaining a patient has a body mass index of approximately 40 or higher; ascertaining the patient does not have a history or current diagnosis of type 2 diabetes mellitus; ascertaining if the patient has a history or current diagnosis of myocardial infarction, stroke, chronic heart failure, hypertension, obstructive sleep apnea, or non-alcoholic fatty liver disease; if the patient has a history or current diagnosis of myocardial infarction, stroke, chronic heart failure, hypertension, obstructive sleep apnea, or non-alcoholic fatty liver disease, initiate dual therapy generic anti-obesity medications or medications targeting both Glucagon-Like Peptide-1 (GLP-1) and Gastric Inhibitory Polypeptide (GIP) receptors and a lifestyle modification; if the patient does not have a history or current diagnosis of myocardial infarction, stroke, chronic heart failure, hypertension, obstructive sleep apnea, or non-alcoholic fatty liver disease, initiate dual therapy generic anti-obesity medications or medications targeting both Glucagon-Like Peptide-1 (GLP-1) and Gastric Inhibitory Polypeptide (GIP) receptors and a lifestyle modification; and re-evaluating the patient and updating a medical treatment of the patient based on the re-evaluating.

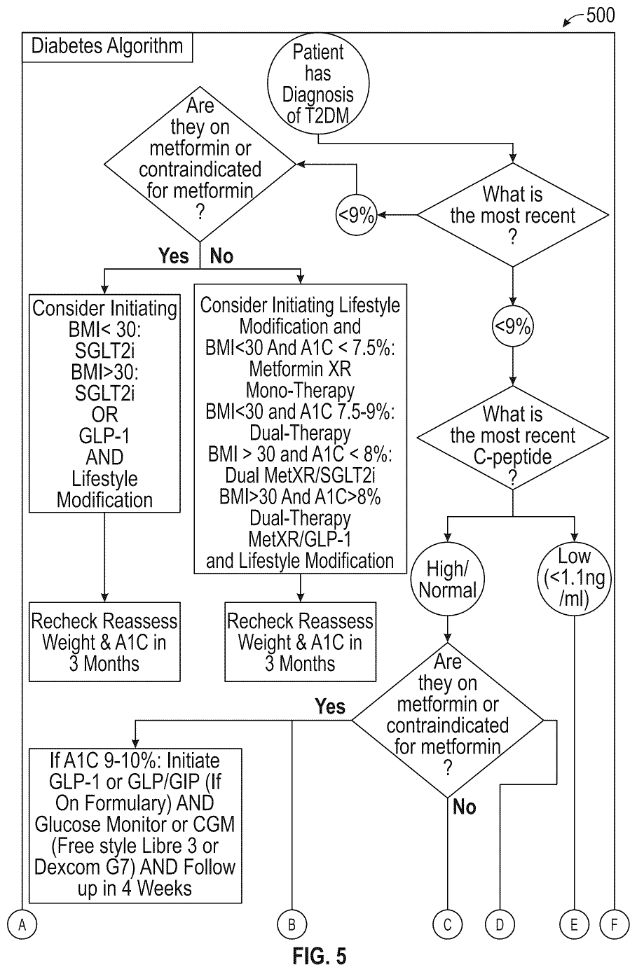

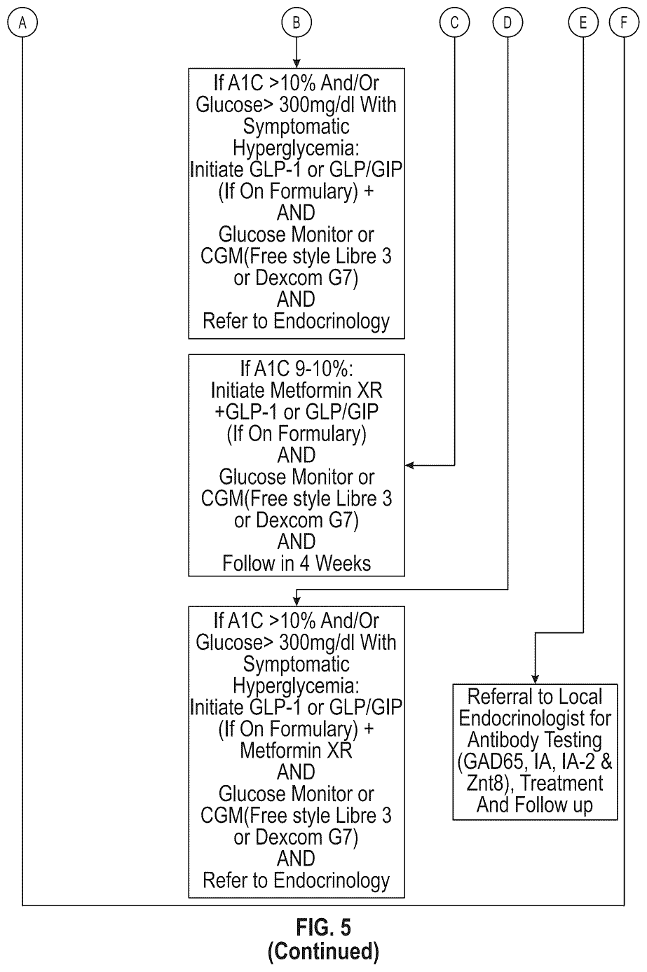

FIG. 5 illustrates a method 500 of pharmacotherapeutic intervention for treatment of obesity targeting multiple dedicated neurophysiological mechanisms of appetite and weight regulation to achieve significant and durable weight loss for a patient with a diagnosis of type 2 diabetes mellitus, according to embodiments of the present technology. FIG. 5 illustrates the method 500 of pharmacotherapeutic intervention for treatment of obesity targeting multiple dedicated neurophysiological mechanisms of appetite and weight regulation to achieve significant and durable weight loss for a patient, the method comprising: ascertaining a patient has a diagnosis of type 2 diabetes mellitus; determining a recent hemoglobin A1c level to be either below 9% or greater than 9% for the patient; if greater than 9%, determining if the most recent c-peptide level is normal or high; determining if the patient is on metformin or contraindicated for metformin; if the patient is on metformin or contraindicated for metformin, and the recent hemoglobin A1c level is between 9% to 10%, initiate medications targeting both Glucagon-Like Peptide-1 (GLP-1) and Gastric Inhibitory Polypeptide (GIP) receptors and use a glucose monitor and follow-up in 4 weeks; if the patient is on metformin or contraindicated for metformin, and the recent hemoglobin A1c level is greater than 10% and/or the glucose level is greater than 300 mg/dl with symptomatic hyperglycemia, initiate medications targeting both Glucagon-Like Peptide-1 (GLP-1) and Gastric Inhibitory Polypeptide (GIP) receptors and use a glucose monitor and refer to endocrinology; if the patient has the recent hemoglobin A1c level is greater than 10% and/or the glucose level is greater than 300 mg/dl with symptomatic hyperglycemia, initiate medications targeting both Glucagon-Like Peptide-1 (GLP-1) and Gastric Inhibitory Polypeptide (GIP) receptors and Metformin XR and use a glucose monitor and refer to endocrinology; if the patient has the recent hemoglobin A1c level is between 9% to 10%, initiate Metformin XR or medications targeting both Glucagon-Like Peptide-1 (GLP-1) and Gastric Inhibitory Polypeptide (GIP) receptors and use a glucose monitor and follow-up in 4 weeks; if less than 9%, determining if the most recent c-peptide level is normal or high; determining if the patient is on metformin or contraindicated for metformin; if the patient is on metformin or contraindicated for metformin, initiate Sodium-Glucose Cotransporter-2 Inhibitor if a body mass index of the patient is less than 30 and recheck and reassess weight and A1C in 3 months and a lifestyle change; if the patient is on metformin or contraindicated for metformin, initiate Sodium-Glucose Cotransporter-2 Inhibitor or medications targeting both Glucagon-Like Peptide-1 (GLP-1) and a lifestyle modification if a body mass index of the patient is greater than 30 and recheck and reassess weight and A1C in 3 months and a lifestyle change; if the patient is not on metformin or contraindicated for metformin and BMI is less than 30 and A1C is less than 7.5%, initiate Metformin XR monotherapy and recheck and reassess weight and A1C in 3 months and a lifestyle change; if the patient is not on metformin or contraindicated for metformin and BMI is less than 30 and A1C is between 7.5% and 9%, initiate dual therapy of Metformin XR (assuming they are not contraindicated for it) and Sodium-Glucose Cotransporter-2 Inhibitor and recheck and reassess weight and A1C in 3 months and a lifestyle change; if the patient is not on metformin or contraindicated for metformin and BMI is greater than 30 and A1C is less than 8%, initiate dual therapy of Metformin XR (assuming they are not contraindicated for it) and Sodium-Glucose Cotransporter-2 Inhibitor and recheck and reassess weight and A1C in 3 months and a lifestyle change; and if the patient is not on metformin or contraindicated for metformin and BMI is greater than 30 and A1C is greater than 8%, initiate dual therapy of Metformin XR (assuming they are not contraindicated for it) and medications targeting both Glucagon-Like Peptide-1 (GLP-1) and recheck and reassess weight and A1C in 3 months and a lifestyle change.

In some embodiments continuous glucose monitors (CGMs) may be used, which are used to track glucose levels in patients, particularly those with diabetes. In some embodiments key terms include postprandial glucose excursions, dawn phenomenon, and Somogyi (rebound) effect. For example, Postprandial Glucose Excursions is 2-Hour Post-Meal Glucose: ≥140 mg/dl and Pre-meal to 2-Hour Post-Meal glucose change: ≥50 mg/dl. Dawn Phenomenon is Glucose >140 mg/dL between 2-6 AM that is caused by hormone-induced (cortisol and growth hormone) liver glucose release. Somogyi or Rebound Effect is overnight hypoglycemia that rebounds due to multiple hormone responses (cortisol, glucagon, growth hormone, adrenaline) and tends to normalize or is elevated by morning.

Some embodiments include, recurring trends in hypoglycemia and hyperglycemia. For example, possible causes of hypoglycemia include medications, overnight lows, physical activity, or alcohol consumption. Hyperglycemia possible causes include under-medication (i.e., too little insulin administration), poor compliance, or patterns on weekends, night shifts, or certain meals. Additionally, target glucose ranges and time-in-range goals for patients with type 1 and type 2 diabetes are included below, and recommendations for adjusting treatment based on CGM data. Specifically, emphasis on the importance of first addressing hypoglycemia (low blood sugar), including overnight lows and post-meal drops, and then managing hyperglycemia (high blood sugar) by adjusting medication dosages and reviewing lifestyle factors. The guidance is intended to optimize glucose control and inform therapeutic decisions for patients using CGMs. For example, Time in Range Goals include T2DM: Aim for >90% time in range and T1DM: Aim for >75% time in range. If <50% Time in Range: Consider adding dual or triple therapy for both fasting and postprandial glucose control. If 50%-90% Time in Range: Focus on targeting areas of hyperglycemia based on identified trends. For instance Glucose Goals include Fasting Glucose: 70-110 mg/dL and 2-Hour Post-Meal Glucose (PPG): <140 mg/dL. Some embodiments include Treatment Adjustments including first addressing hypoglycemia such as Overnight Lows: Adjust basal insulin or correction factor (CF); adjust long acting sulfonylurea if present, 1-2 Hour Post-Meal Hypoglycemia: Adjust insulin-to-carb ratio (ICR) or mealtime medications, and Activity or Meal-Skipping-Related Hypoglycemia: Adjust basal insulin or sulfonylurea. Second managing hyperglycemia (high blood sugar) including address Hyperglycemia trends. For instance, Post-Meal Glucose Rise ≥50 mg/dL: Adjust ICR or mealtime medication dosage; review carbohydrate intake, and Elevated Fasting or Pre-Meal Glucose: Adjust CF or basal medication; consider factors like physical activity, stress, sleep, or recent illness.

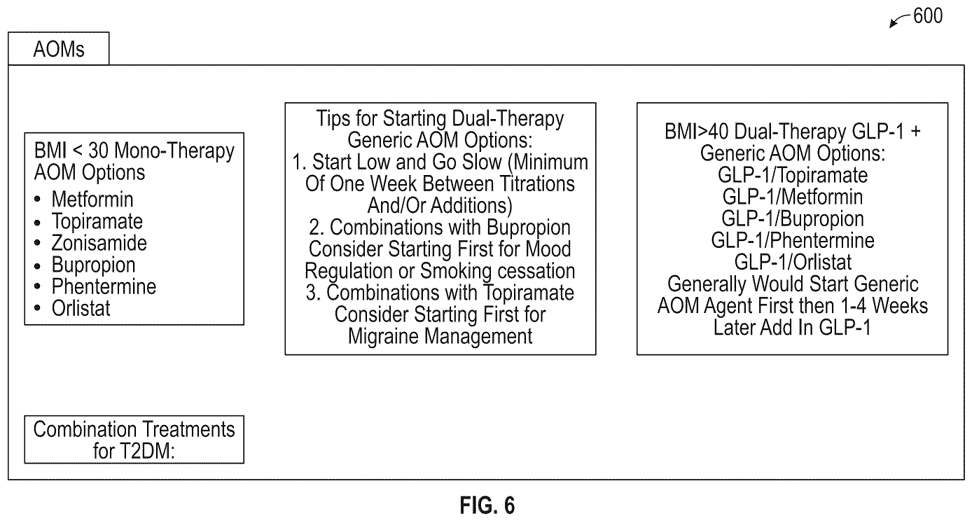

FIG. 6 illustrates dual-therapy and mono-therapy options 600 for a method of pharmacotherapeutic intervention for treatment of obesity targeting multiple dedicated neurophysiological mechanisms of appetite and weight regulation to achieve significant and durable weight loss for a patient, according to embodiments of the present technology. For example, FIG. 6 is an illustration of a flowchart detailing various pharmacotherapeutic options for anti-obesity medications (AOMs) tailored to different body mass index (BMI) categories and specific patient conditions, wherein the method for treating obesity targets multiple neurophysiological mechanisms of appetite and weight regulation, thereby facilitating personalized obesity management, according to various embodiments of the present technology.

According to some embodiments, the flowchart begins with a section outlining mono-therapy AOM options for patients having a BMI less than 30, wherein specific medications including Metformin, Topiramate, Zonisamide, Bupropion, Phentermine, and Orlistat are listed as potential single-agent therapies based on the patient's individual phenotype, medical history, and contraindications, thereby guiding selection of an appropriate mono-therapy agent, according to various embodiments of the present technology.

According to some embodiments, the next section provides tips for initiating dual-therapy generic AOM options, wherein a cautious and gradual approach with a minimum of one week between titrations or additions of medications is recommended, and wherein combinations involving Bupropion are initiated first for patients requiring mood regulation or smoking cessation and combinations involving Topiramate are initiated first for patients with migraine management needs, thereby optimizing therapeutic benefit while minimizing potential side effects, according to various embodiments of the present technology.

According to some embodiments, for patients having a BMI greater than 40, the flowchart outlines dual-therapy options combining glucagon-like peptide-1 (GLP-1) receptor agonists with generic AOMs, wherein combinations include GLP-1 receptor agonists co-administered with Topiramate, Metformin, Bupropion, Phentermine, or Orlistat, and wherein treatment is initiated with a generic AOM agent followed by addition of a GLP-1 receptor agonist after one to four weeks, thereby enhancing treatment efficacy while allowing for patient adaptation to the pharmacological regimen, according to various embodiments of the present technology.

According to some embodiments, additionally, the flowchart includes a section on combination treatments for type 2 diabetes mellitus (T2DM), wherein anti-obesity medications are integrated with other pharmacological agents tailored to the patient's metabolic profile and comorbid conditions, thereby providing a comprehensive and individualized therapeutic approach for patients with coexisting obesity and diabetes, according to various embodiments of the present technology.

Overall, FIG. 6 provides a structured framework for selecting and initiating pharmacotherapeutic interventions for obesity treatment, wherein patients are categorized based on BMI and other clinical factors to facilitate the implementation of personalized treatment plans that target multiple neurophysiological mechanisms, thereby aiming to achieve significant and durable weight loss while addressing associated comorbidities, according to various embodiments of the present technology.

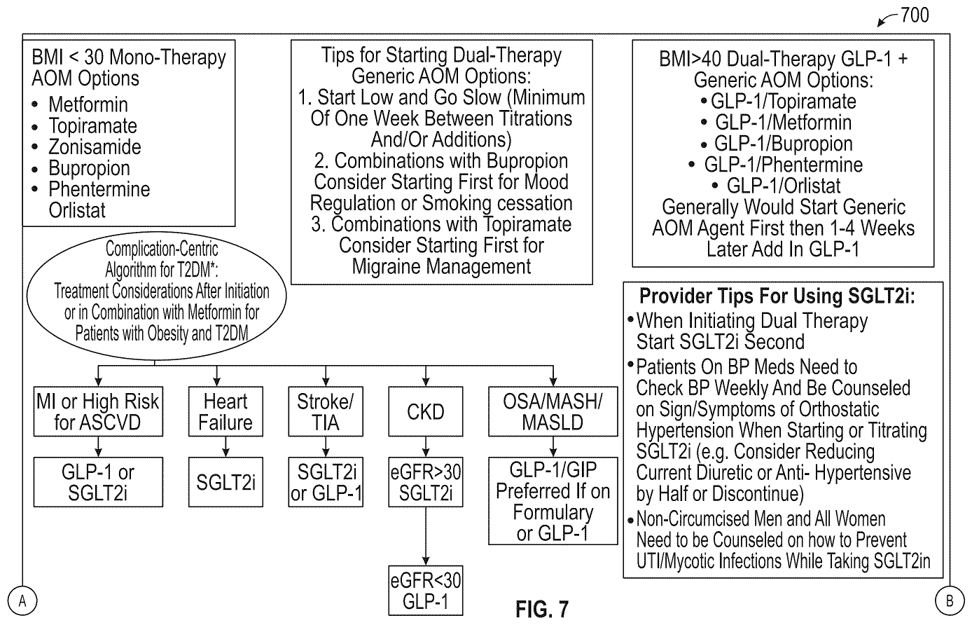

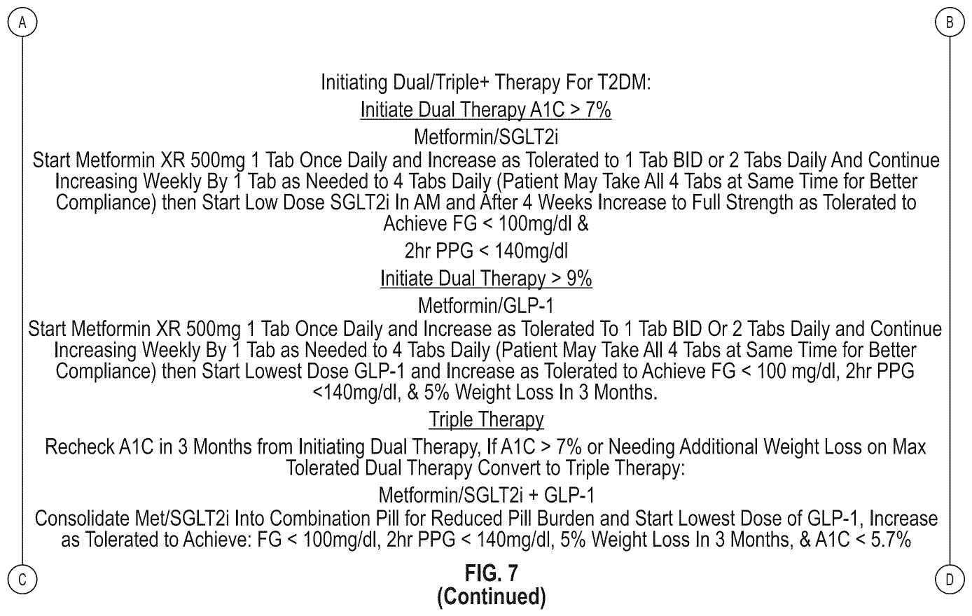

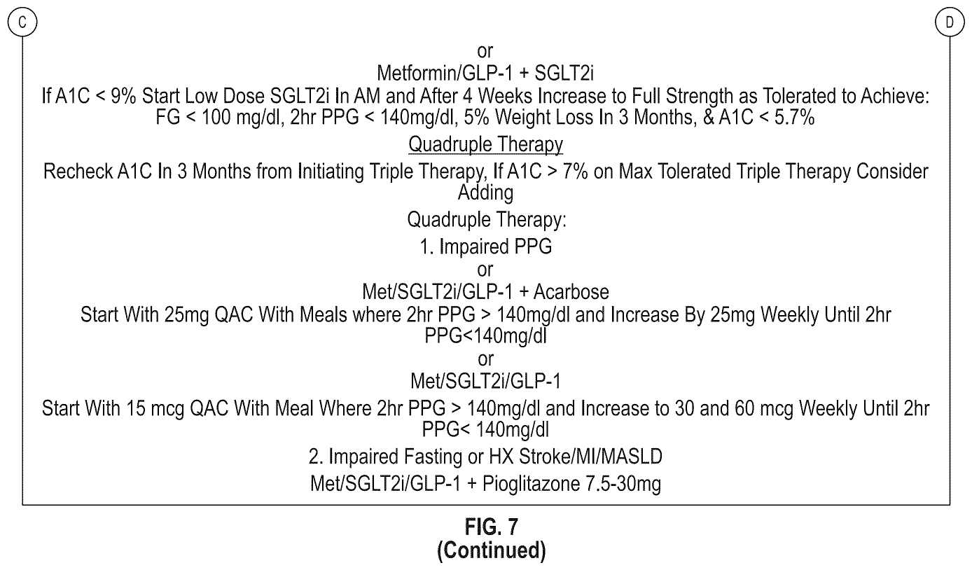

FIG. 7 illustrates dual-therapy and mono-therapy options for a method 700 of pharmacotherapeutic intervention for treatment of obesity targeting multiple dedicated neurophysiological mechanisms of appetite and weight regulation to achieve significant and durable weight loss for a patient, according to embodiments of the present technology.

According to some embodiments, FIG. 7 is a comprehensive method 700 detailing dual-therapy and mono-therapy options for pharmacotherapeutic interventions in the treatment of obesity wherein the table is structured to provide healthcare providers with a systematic approach to selecting and initiating appropriate anti-obesity medications (AOMs) based on patient-specific factors, including body mass index (BMI), comorbid conditions, and treatment goals, and wherein the figure further includes guidance for managing patients with type 2 diabetes mellitus (T2DM) and other obesity-related complications, according to various embodiments of the present technology.

In some embodiments, the table is divided into several sections, each addressing specific therapeutic strategies. Some embodiments include Mono-Therapy AOM Options for Patients with BMI less than 30 wherein the section lists potential single-agent therapies, including Metformin, Topiramate, Zonisamide, Bupropion, Phentermine, and Orlistat, such that each medication is selected based on the patient's individual phenotype, medical history, and contraindications, thereby allowing for personalized treatment tailored to the patient's specific needs.

According to some embodiments, advantages for Initiating Dual-Therapy Generic AOM Options include practical recommendations for starting dual-therapy regimens with a cautious and gradual approach, including a minimum of one week between titrations or additions of medications, and wherein specific combinations are suggested based on additional patient needs (e.g., mood regulation, smoking cessation, or migraine management), such that combinations involving Bupropion are recommended for mood regulation or smoking cessation and combinations involving Topiramate are suggested for patients with migraines.

According to some embodiments, dual-Therapy Options for Patients with BMI greater than 40 includes dual-therapy options that combine glucagon-like peptide-1 (GLP-1) receptor agonists with generic AOMs, such as Topiramate, Metformin, Bupropion, Phentermine, or Orlistat, such that treatment is initiated with a generic AOM agent and a GLP-1 receptor agonist is added after one to four weeks to enhance efficacy and allow for patient adaptation.

According to some embodiments, Provider Tips for Using Sodium-Glucose Cotransporter-2 Inhibitors (SGLT2i) wherein the section offers guidance for initiating dual therapy involving SGLT2 inhibitors as the second agent, including recommendations for weekly blood pressure monitoring in patients on antihypertensive medications and counseling to prevent urinary tract or mycotic infections, particularly in non-circumcised men and women.

According to some embodiments, Complication-Centric Algorithm for T2DM provides a decision-making framework for selecting pharmacotherapeutic interventions based on specific complications, such as myocardial infarction (MI), heart failure, stroke, chronic kidney disease (CKD), obstructive sleep apnea (OSA), and metabolic-associated steatohepatitis (MASH) or metabolic-associated steatotic liver disease (MASLD), such that GLP-1 receptor agonists or SGLT2 inhibitors are recommended for patients with a history of MI or high risk for atherosclerotic cardiovascular disease (ASCVD), and SGLT2 inhibitors are preferred for heart failure or CKD.

According to some embodiments, administering Dual/Triple/Quadruple Therapy for T2DM wherein the section outlines stepwise protocols for initiating and escalating therapy, beginning with dual therapy comprising Metformin and SGLT2i or GLP-1 receptor agonists and progressing to triple or quadruple therapy if additional glycemic control or weight loss is required, with specific dosing and titration guidelines provided to achieve target fasting glucose (FG), postprandial glucose (PPG), weight loss, and glycated hemoglobin (A1C) levels.

Some embodiments include using of SGLT2 inhibitors. Overall, the present technology includes guidance on the safe and effective use of SGLT2 inhibitors in the management of diabetes and obesity. SGLT2 inhibitors are a class of medications that lower blood sugar and assist with weight management by promoting the excretion of excess glucose through urine. Because of the excretion of excess glucose through urine patients taking SGLT2 inhibitors have an increased risk of urinary tract and yeast infections associated with these medications. Accordingly, the present technology includes practical advice to patients for prevention, such as drinking plenty of water, maintaining a balanced diet, and keeping the genital area clean and dry. Special instructions are given for uncircumcised men regarding hygiene. Furthermore, it is important to monitor for symptoms of infection and contacting a healthcare provider if concerns arise. Specifically, patients are advised to drink Plenty of Water: Aim to drink at least 64 ounces of water daily in order to help dilute urine and reduce the amount of sugar in a patient's urine, lowering the risk of infections. Specifically, patients are advised to Eat a Balanced Diet: Try to avoid meals that are high in carbohydrates, as they can increase the sugar in a patient's urine. Specifically, patients are advised to Stay Dry: After you urinate, make sure to dry a patient's skin. For example, leaving urine on skin may lead to yeast infections. Specifically, patients that are Uncircumcised Men are advised to be sure to pull back the foreskin when urinating and clean the area daily to keep it clean and dry. Accordingly, FIG. 7 emphasizes the importance of re-evaluating patients at regular intervals, typically on a quarterly basis, to assess treatment efficacy and make necessary adjustments, thereby ensuring that the pharmacotherapeutic intervention remains aligned with the patient's evolving clinical needs and treatment goals, according to various embodiments.

In some embodiments, an Artificially Intelligent (AI) component may be used to offer personalized pharmacotherapy phenotyping, powered by advanced algorithms trained on a range of obesity-related medication treatment protocols based upon large language models (LLMs). This AI system can be retrained based on outcomes to further improve its guidance.

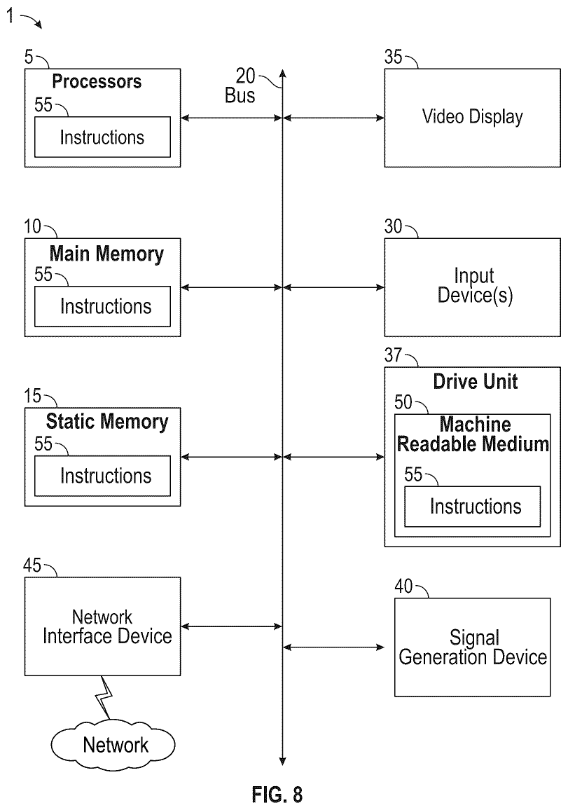

FIG. 8 illustrates an exemplary computer system that may be used to implement embodiments of the present disclosure. FIG. 8 is a diagrammatic representation of an example machine in the form of a computer system 1, within which a set of instructions for causing the machine to perform any one or more of the methodologies discussed herein may be executed. In various example embodiments, the machine operates as a standalone device or may be connected (e.g., networked) to other machines. In a networked deployment, the machine may operate in the capacity of a server or a client machine in a server-client network environment, or as a peer machine in a peer-to-peer (or distributed) network environment. The machine may be a personal computer (PC), a tablet PC, a set-top box (STB), a personal digital assistant (PDA), a cellular telephone, a portable music player (e.g., a portable hard drive audio device such as a Moving Picture Experts Group Audio Layer 3 (MP3) player), a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The computer system 1 includes a processor or multiple processor(s) 5 (e.g., a central processing unit (CPU), a graphics processing unit (GPU), or both), and a main memory 10 and static memory 15, which communicate with each other via a bus 20. The computer system 1 may further include a video display 35 (e.g., a liquid crystal display (LCD)). The computer system 1 may also include an alpha-numeric input device(s) 30 (e.g., a keyboard), a cursor control device (e.g., a mouse), a voice recognition or biometric verification unit (not shown), a drive unit 37 (also referred to as disk drive unit), a signal generation device 40 (e.g., a speaker), and a network interface device 45. The computer system 1 may further include a data encryption module (not shown) to encrypt data.

The drive unit 37 includes a computer or machine-readable medium 50 on which is stored one or more sets of instructions and data structures (e.g., instructions 55) embodying or utilizing any one or more of the methodologies or functions described herein. The instructions 55 may also reside, completely or at least partially, within the main memory 10 and/or within the processor(s) 5 during execution thereof by the computer system 1. The main memory 10 and the processor(s) 5 may also constitute machine-readable media.

The instructions 55 may further be transmitted or received over a network via the network interface device 45 utilizing any one of a number of well-known transfer protocols (e.g., Hyper Text Transfer Protocol (HTTP)). While the machine-readable medium 50 is shown in an example embodiment to be a single medium, the term “computer-readable medium” should be taken to include a single medium or multiple media (e.g., a centralized or distributed database and/or associated caches and servers) that store the one or more sets of instructions. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, and carrier wave signals. Such media may also include, without limitation, hard disks, floppy disks, flash memory cards, digital video disks, random access memory (RAM), read only memory (ROM), and the like. The example embodiments described herein may be implemented in an operating environment comprising software installed on a computer, in hardware, or in a combination of software and hardware.

Where appropriate, the functions described herein can be performed in one or more of hardware, software, firmware, digital components, or analog components. For example, the encoding and or decoding systems can be embodied as one or more application specific integrated circuits (ASICs) or microcontrollers that can be programmed to carry out one or more of the systems and procedures described herein. Certain terms are used throughout the description and claims refer to particular system components. As one skilled in the art will appreciate, components may be referred to by different names. This document does not intend to distinguish between components that differ in name, but not function.

One skilled in the art will recognize that the Internet service may be configured to provide Internet access to one or more computing devices that are coupled to the Internet service, and that the computing devices may include one or more processors, buses, memory devices, display devices, input/output devices, and the like. Furthermore, those skilled in the art may appreciate that the Internet service may be coupled to one or more databases, repositories, servers, and the like, which may be utilized in order to implement any of the embodiments of the disclosure as described herein.

Aspects of the present technology are described above with reference to flowchart illustrations and/or block diagrams of methods, apparatus (systems), and computer program products according to embodiments of the present technology. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

Various modifications and alterations of the invention will become apparent to those skilled in the art without departing from the spirit and scope of the invention, which is defined by the accompanying claims. It should be noted that steps recited in any method claims below do not necessarily need to be performed in the order that they are recited. Those of ordinary skill in the art will recognize variations in performing the steps from the order in which they are recited. In addition, the lack of mention or discussion of a feature, step, or component provides the basis for claims where the absent feature or component is excluded by way of a proviso or similar claim language.

While various embodiments of the present invention have been described above, it should be understood that they have been presented by way of example only, and not of limitation. The various diagrams may depict an example architectural or other configuration for the invention, which is done to aid in understanding the features and functionality that may be included in the invention. The invention is not restricted to the illustrated example architectures or configurations, but the desired features may be implemented using a variety of alternative architectures and configurations. Indeed, it will be apparent to one of skill in the art how alternative functional, logical, or physical partitioning and configurations may be implemented to implement the desired features of the present invention. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the invention, whether or not such embodiments are described and whether or not such features are presented as being a part of a described embodiment. Thus the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the such as; the term “example” is used to provide exemplary instances of the item in discussion, not an exhaustive or limiting list thereof; the terms “a” or “an” should be read as meaning “at least one,” “one or more” or the such as; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Hence, where this document refers to technologies that would be apparent or known to one of ordinary skill in the art, such technologies encompass those apparent or known to the skilled artisan now or at any time in the future.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof unless limitation to the singular is explicitly stated.

The presence of broadening words and phrases such as “one or more,” “at least,” “but not limited to” or other such as phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, may be combined in a single package or separately maintained and may further be distributed across multiple locations.

Additionally, the various embodiments set forth herein are described in terms of exemplary block diagrams, flow charts, and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration.

The previous description of the disclosed embodiments is provided to enable any patient skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects. The Abstract of the Disclosure is provided to comply with 37 C.F.R. § 1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, various features may be grouped together to streamline the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

Thus, the technology for systems and methods of pharmacotherapeutic intervention for treatment of obesity targeting multiple dedicated neurophysiological mechanisms of appetite and weight regulation to achieve significant and durable weight loss for a patient is disclosed. Although embodiments have been described with reference to specific example embodiments, it will be evident that various modifications and changes can be made to these example embodiments without departing from the broader spirit and scope of the present application. Accordingly, the specification and drawings are to be regarded in an illustrative rather than a restrictive sense.

What is claimed is:

1. A method of selecting a pharmacotherapeutic intervention for treatment of obesity targeting multiple dedicated neurophysiological mechanisms of appetite and weight regulation to achieve significant and durable weight loss for a classified patient with a Body Mass Index (BMI) of 27 to 29.9, the method comprising:
receiving patient data comprising at least body-mass-index (BMI), medical history, laboratory data, and prior medication response;
classifying a patient into a predefined BMI range using the patient data, the predefined BMI range being a Body Mass Index (BMI) of 27 to 29.9 thereby the classified patient being classified as overweight;
determining, for the classified patient, an absence of type-2 diabetes mellitus using the patient data;
receiving a lifestyle modification confirmation for the classified patient, the lifestyle modification confirmation for the classified patient being that the classified patient makes a lifestyle modification;
administering a therapeutically effective amount of a non-glucagon-like peptide-1 (non-GLP-1) agonist anti-obesity medication to the classified patient, the administering the therapeutically effective amount of the non-GLP-1 agonist anti-obesity medication to the classified patient being a particular prophylaxis targeting multiple dedicated neurophysiological mechanisms that is an obesity treatment for patients having a Body Mass Index (BMI) of approximately 27 to 29.9 in combination with the lifestyle modification confirmation; and
re-evaluating the classified patient and updating a medical treatment plan of the classified patient based on the re-evaluating.

2. The method of claim 1, wherein the non-glucagon-like peptide-1 (non-GLP-1) agonist anti-obesity medication is at least one of: metformin, topiramate, zonisamide, bupropion, naltrexone, phentermine, and orlistat.

3. The method of claim 1, wherein the lifestyle modification confirmation comprises confirmation that the classified patient adopts a hypocaloric diet or increases physical activity to a target of at least 150 minutes per week.

4. The method of claim 1, wherein re-evaluating the classified patient comprises measuring a body weight of the classified patient and reassessing body-mass-index (BMI) after a three-month interval.

5. The method of claim 1, wherein determining the absence of type-2 diabetes mellitus comprises analyzing a glycated hemoglobin (HbA1c) level of the classified patient to confirm that the glycated hemoglobin (HbA1c) level is below 6.5%.

6. The method of claim 1, further comprising generating a clinician-facing report that lists a selected pharmacotherapeutic regimen, recommended dosing schedule, and a scheduled re-evaluation date.

7. A method of selecting a pharmacotherapeutic intervention for treatment of obesity targeting multiple dedicated neurophysiological mechanisms of appetite and weight regulation to achieve significant and durable weight loss for a classified patient with a Body Mass Index (BMI) of 30 to 34.9, the method comprising:
receiving patient data comprising at least body-mass-index (BMI), medical history, laboratory data, and prior medication response;
classifying a patient into a predefined BMI range using the patient data, the predefined BMI range being a Body Mass Index (BMI) of 30 to 34.9 thereby the classified patient being classified in class 1 of obesity;
determining, for the classified patient, an absence of type-2 diabetes mellitus using the patient data;
determining, for the classified patient, an absence of history or current diagnosis of myocardial infarction, stroke, chronic heart failure, hypertension, obstructive sleep apnea, or non-alcoholic fatty liver disease;
receiving a lifestyle modification confirmation for the classified patient, the lifestyle modification confirmation for the classified patient being that the classified patient makes a lifestyle modification;
administering a therapeutically effective amount of dual therapy non-glucagon-like peptide-1 (non-GLP-1) agonist anti-obesity medications to the classified patient, the administering the therapeutically effective amount of the dual therapy non-GLP-1 agonist anti-obesity medications to the classified patient being a particular prophylaxis targeting multiple dedicated neurophysiological mechanisms that is an obesity treatment for patients having a Body Mass Index (BMI) of approximately 30 to 34.9 in combination with the lifestyle modification confirmation; and
re-evaluating the patient and updating a medical treatment of the patient based on the re-evaluating.

8. The method of claim 7, wherein the dual therapy non-glucagon-like peptide-1 (non-GLP-1) agonist anti-obesity medications comprise a combination selected from the group consisting of metformin and bupropion, metformin and topiramate, bupropion and topiramate, bupropion and naltrexone, phentermine and topiramate, and phentermine and metformin.

9. The method of claim 7, wherein the lifestyle modification comprises adoption of an increase in physical activity for the classified patient to a target of at least 150 minutes per week.

10. The method of claim 7, wherein re-evaluating the classified patient comprises measuring a body weight of the classified patient and reassessing body-mass-index (BMI) after a three-month interval.

11. A method of selecting a pharmacotherapeutic intervention for treatment of obesity targeting multiple dedicated neurophysiological mechanisms of appetite and weight regulation to achieve significant and durable weight loss for a classified patient with a Body Mass Index (BMI) of 35 to 39.9, the method comprising:
receiving patient data comprising at least body-mass-index (BMI), medical history, laboratory data, and prior medication response;
classifying a patient into a predefined BMI range using the patient data, the predefined BMI range being a Body Mass Index (BMI) of 35 to 39.9 thereby the classified patient being classified in class 2 of obesity;
determining, for the classified patient, an absence of type-2 diabetes mellitus using the patient data;
determining, for the classified patient, an absence of history or current diagnosis of myocardial infarction, stroke, chronic heart failure, hypertension, obstructive sleep apnea, or non-alcoholic fatty liver disease;
receiving a lifestyle modification confirmation for the classified patient, the lifestyle modification confirmation for the classified patient being that the classified patient makes a lifestyle modification;
administering a therapeutically effective amount of dual therapy non-glucagon-like peptide-1 (non-GLP-1) agonist anti-obesity medications to the classified patient, the administering the therapeutically effective amount of the dual therapy non-GLP-1 agonist anti-obesity medications to the classified patient being a particular prophylaxis targeting multiple dedicated neurophysiological mechanisms that is an obesity treatment for patients having a Body Mass Index (BMI) of approximately 30 to 34.9 in combination with the lifestyle modification confirmation; and
re-evaluating the patient and updating a medical treatment of the patient based on the re-evaluating.

12. The method of claim 11, wherein the dual therapy non-glucagon-like peptide-1 (non-GLP-1) agonist anti-obesity medications comprise a combination selected from the group consisting of metformin and bupropion, metformin and topiramate, bupropion and topiramate, bupropion and naltrexone, phentermine and topiramate, and phentermine and metformin.

13. The method of claim 11, wherein the lifestyle modification comprises adoption of an increase in physical activity for the classified patient to a target of at least 150 minutes per week.

14. The method of claim 11, wherein re-evaluating the classified patient comprises measuring a body weight of the classified patient and reassessing body-mass-index (BMI) after a three-month interval.

15. A method of selecting a pharmacotherapeutic intervention for treatment of obesity targeting multiple dedicated neurophysiological mechanisms of appetite and weight regulation to achieve significant and durable weight loss for a classified patient with a Body Mass Index (BMI) of 40 or higher, the method comprising:
receiving patient data comprising at least body-mass-index (BMI), medical history, laboratory data, and prior medication response;
classifying a patient into a predefined BMI range using the patient data, the predefined BMI range being a Body Mass Index (BMI) of 40 or higher thereby the classified patient being classified in class 3 of obesity;
determining, for the classified patient, an absence of type-2 diabetes mellitus using the patient data;
receiving a lifestyle modification confirmation for the classified patient, the lifestyle modification confirmation for the classified patient being that the classified patient makes a lifestyle modification;
determining, for the classified patient, a history or current diagnosis of myocardial infarction, stroke, chronic heart failure, hypertension, obstructive sleep apnea, or non-alcoholic fatty liver disease;
administering a therapeutically effective amount of dual therapy non-glucagon-like peptide-1 (non-GLP-1) agonist anti-obesity medications or Gastric Inhibitory Polypeptide (GIP) agonist to the classified patient, the administering the therapeutically effective amount of the dual therapy non-GLP-1 agonist anti-obesity medications or Gastric Inhibitory Polypeptide (GIP) agonist being a particular prophylaxis targeting multiple dedicated neurophysiological mechanisms that is an obesity treatment for patients having a Body Mass Index (BMI) of 40 or higher in combination with the lifestyle modification confirmation; and
re-evaluating the patient and updating a medical treatment of the patient based on the re-evaluating.

16. The method of claim 15, wherein the dual therapy non-glucagon-like peptide-1 (non-GLP-1) agonist anti-obesity medications comprise a combination selected from the group consisting of metformin and bupropion, metformin and topiramate, bupropion and topiramate, bupropion and naltrexone, phentermine and topiramate, and phentermine and metformin.

17. The method of claim 15, wherein the lifestyle modification comprises adoption of an increase in physical activity for the classified patient to a target of at least 150 minutes per week.

18. The method of claim 15, wherein re-evaluating the classified patient comprises measuring a body weight of the classified patient and reassessing body-mass-index (BMI) after a three-month interval.

19. A method of pharmacotherapeutic intervention for treatment of obesity targeting multiple dedicated neurophysiological mechanisms of appetite and weight regulation to achieve significant and durable weight loss for a patient that has a diagnosis of type 2 diabetes mellitus, the method comprising:
ascertaining a patient has a diagnosis of type 2 diabetes mellitus;
determining a recent hemoglobin A1c level to be either below 9% or greater than 9% for the patient;
determining a recent hemoglobin A1c level is greater than 9%;
determining if a most recent c-peptide level is either low or normal/high;
determining the most recent c-peptide level is normal/high;
determining if the patient is on metformin or contraindicated for metformin;
determining the recent hemoglobin A1c level is between 9% to 10%; and
administering a therapeutically effective amount of medications to the patient targeting either Glucagon-Like Peptide-1 (GLP-1) receptors or both Glucagon-Like Peptide-1 (GLP-1) and Gastric Inhibitory Polypeptide (GIP) receptors, the administering being a particular prophylaxis targeting multiple dedicated neurophysiological mechanisms that is a specific obesity treatment for patients having a diagnosis of type 2 diabetes mellitus and a recent hemoglobin A1c level between 9% to 10%.
